(19) United States
(12) Patent Application Publication
(10) Pub. No.: US 2021/0145407 A1
(43) Pub. Date: May 20, 2021

(54) MEDICAL IMAGE DISPLAY APPARATUS, REGION DISPLAY METHOD, AND REGION DISPLAY PROGRAM

(71) Applicant: Konica Minolta, Inc., Tokyo (JP)

(72) Inventor: Kazuya TAKAGI, Tokyo (JP)

(21) Appl. No.: 17/060,926

(22) Filed: Oct. 1, 2020

(30) Foreign Application Priority Data
Nov. 20, 2019 (JP) 2019-209656

(51) Int. Cl. A61B 8/00; A61B 8/12

(52) U.S. Cl. CPC A61B 8/461; A61B 8/12; A61B 8/467

(57) ABSTRACT

A medical image display apparatus includes: a hardware processor that determines a low-luminance region on a medical image related to a biotissue inside a subject based on a luminance value of a pixel constituting the medical image, the low-luminance region having a relatively low luminance and being sandwiched between a plurality of high-luminance regions having a relatively high luminance; and a display that displays the determined low-luminance region on the medical image in a display mode different from that of a region other than the low-luminance region.

The entire disclosure of Japanese patent Application No. 2019-209656, filed on Nov. 20, 2019, is incorporated herein by reference in its entirety.

### BACKGROUND

### Technological Field

The present invention relates to a medical image display apparatus, a region display method, and a region display program.

### Description of the Related Art

An ultrasonic diagnostic apparatus has been conventionally known as one of medical image diagnostic apparatuses. The ultrasonic diagnostic apparatus visualizes the shape, property, or dynamics of the inside of a subject as an ultrasonic image by transmitting an ultrasonic wave to the subject, receiving the reflected wave, and performing predetermined signal processing on a signal of the received reflected wave.

For example, ultrasonic diagnostic apparatuses disclosed in WO 2017/138086 and JP 2006-20800 A visualize the inside of a subject by detecting a moving region by the difference between frames of an ultrasonic image and performing highlighting of changing the luminance value or color of the region. Such an ultrasonic diagnostic apparatus can acquire an ultrasonic image by a simple operation of applying an ultrasonic probe on a body surface or inserting the ultrasonic probe into a body cavity, so that the ultrasonic diagnostic apparatus is safe, and puts less burden on a subject.

Treatment methods (e.g., HydroRelease) of injecting liquid medicine or saline into body for reducing pain such as aching pain have recently attracted attention. In the treatment methods such as HydroRelease, liquid medicine or saline is often injected into a body with reference to an ultrasonic image obtained by an ultrasonic diagnostic apparatus (echo guide). At this time, the liquid medicine or saline is injected while the expansion of a region into which the liquid medicine or saline is injected is visually recognized.

With poor anatomical knowledge for an ultrasonic image, however, it is unfortunately difficult to visually recognize the expansion of a region into which liquid medicine or saline is injected on an ultrasonic image. The ultrasonic diagnostic apparatuses disclosed in WO 2017/138086 and JP 2006-20800 A can highlight a region expanded by injection of liquid medicine or saline. These ultrasonic diagnostic apparatuses, however, also highlight other regions moved by the injection of the liquid medicine or saline, and a region that interests a user is buried in the other regions. It is thus difficult to determine the region that interests the user.

### SUMMARY

An object of the invention is to provide a medical image display apparatus, a region display method, and a region display program capable of improving the visibility of a region that interests a user.

To achieve the abovementioned object, according to an aspect of the present invention, a medical image display apparatus reflecting one aspect of the present invention comprises: a hardware processor that determines a low-luminance region on a medical image related to a biotissue inside a subject based on a luminance value of a pixel constituting the medical image, the low-luminance region having a relatively low luminance and being sandwiched between a plurality of high-luminance regions having a relatively high luminance; and a display that displays the determined low-luminance region on the medical image in a display mode different from that of a region other than the low-luminance region.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 illustrates the appearance of an ultrasonic diagnostic apparatus according to an embodiment of the invention;

FIG. 2 is a block diagram illustrating a main part of the ultrasonic diagnostic apparatus in FIG. 1;

FIG. 3 is a flowchart illustrating a region display method according to the embodiment of the invention;

FIG. 4 is a flowchart illustrating a high-luminance search in FIG. 3;

FIG. 5A schematically illustrates an ultrasonic image before highlighting;

FIG. 5B schematically illustrates an ultrasonic image that has been subject to a high-luminance search in an image upward direction;

FIG. 5C schematically illustrates an ultrasonic image that has been subject to a high-luminance search in an image downward direction;

FIG. 5D schematically illustrates an ultrasonic image in which a low-luminance region determined based on a result of high-luminance searches in two directions is highlighted;

FIG. 6 is a flowchart illustrating a variation of the region display method according to the embodiment of the invention;

FIG. 7A schematically illustrates an ultrasonic image in which a low-luminance region is highlighted at a time t1 immediately before a time t2;

FIG. 7B schematically illustrates an ultrasonic image in which a low-luminance region is highlighted at the time t2;

FIG. 7C schematically illustrates an ultrasonic image in which a changed region is highlighted, the changed region being a region, in which the luminance value has been changed between the time t1 and the time t2, in the low-luminance region;

FIG. 8 schematically illustrates a screen of a display in which an ultrasonic image and an operation region are displayed, a low-luminance region being highlighted in the ultrasonic image, the operation region being used for changing highlight gain and color;

FIG. 9 schematically illustrates a screen of the display in which an ultrasonic image before highlighting and an ultrasonic image after a low-luminance region is highlighted are displayed side by side; and

FIG. 10 schematically illustrates a screen of the display in which an area information display region and a seek bar are displayed, the area information display region indicating an area value of a highlighted low-luminance region, the seek bar relating to the presently displayed ultrasonic image.

### DETAILED DESCRIPTION OF EMBODIMENTS

Hereinafter, one or more embodiments of the present invention will be described with reference to the drawings. However, the scope of the invention is not limited to the disclosed embodiments. Although an ultrasonic diagnostic apparatus that displays an ultrasonic image being a medical image is described below as one example of medical image display apparatuses, the invention can be applied to an apparatus for generating a medical image based on a signal in accordance with a biotissue inside a subject, for example, an X-ray imaging apparatus.

<Configuration of Ultrasonic Diagnostic Apparatus>

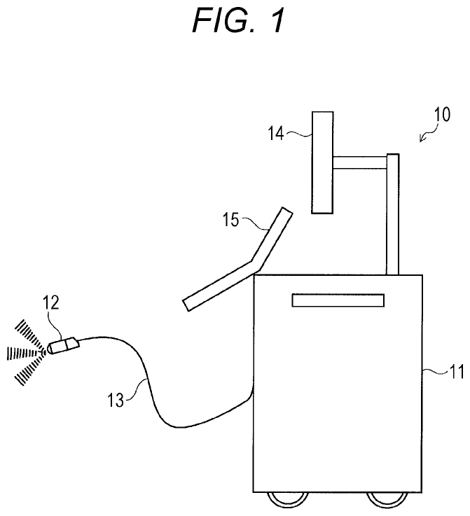

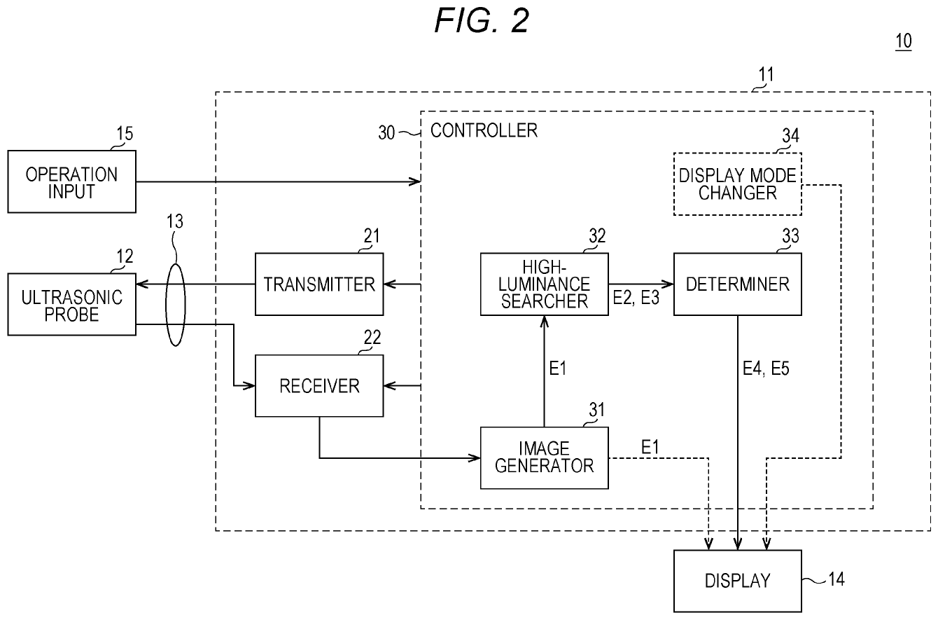

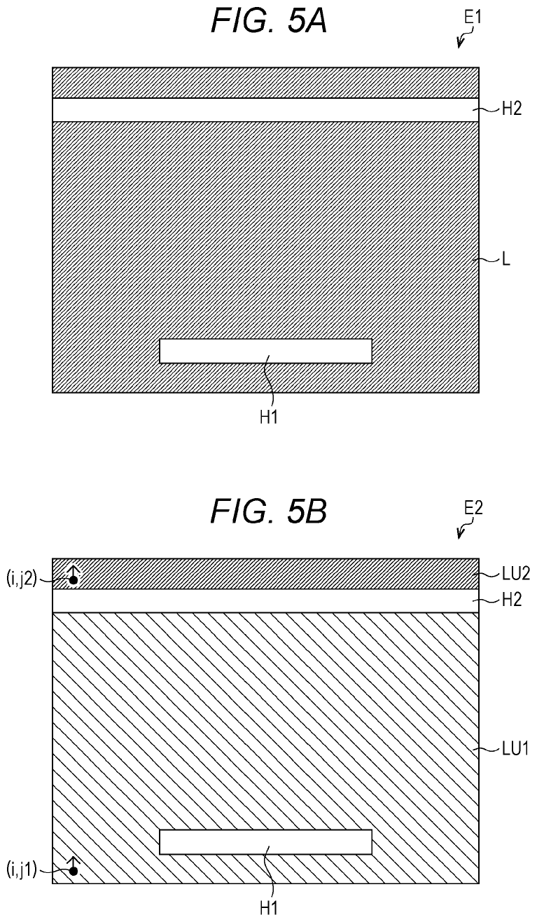

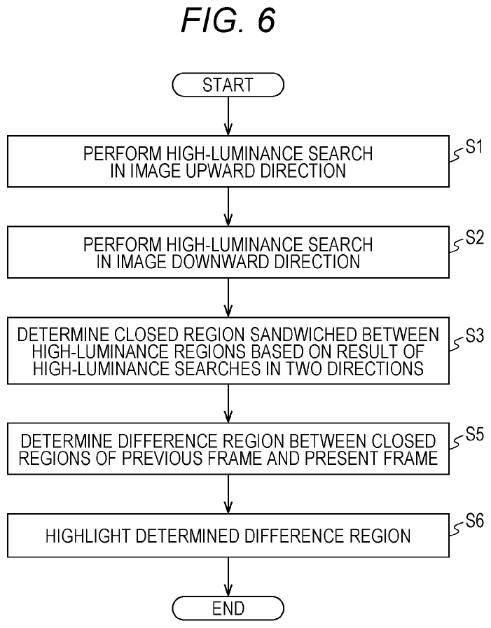

FIG. 1 illustrates the appearance of an ultrasonic diagnostic apparatus 10 according to the embodiment. FIG. 2 is a block diagram illustrating a main part of the ultrasonic diagnostic apparatus 10 in FIG. 1. Signs E1 to E5 in FIG. 2 will be described later in FIGS. 5A to 5D and 7A to 7C.

The ultrasonic diagnostic apparatus 10 is used for visualizing the shape, property, and dynamics of a biotissue inside a subject such as a living body in an ultrasonic image (medical image) and diagnosing the image.

As illustrated in FIG. 1, the ultrasonic diagnostic apparatus 10 includes an apparatus body 11, an ultrasonic probe 12, a cable 13, a display 14, and an operation input 15. The apparatus body 11 and the ultrasonic probe 12 are connected via the cable 13. The ultrasonic probe 12 may be connected to the apparatus body 11 by wireless communication.

The ultrasonic probe 12 functions as an acoustic sensor that transmits an ultrasonic wave to a subject, receives an ultrasonic echo reflected from the subject, converts the ultrasonic echo into an electric signal (reception signal), and transmits the electric signal to the apparatus body 11.

The ultrasonic probe 12 includes a plurality of oscillators including a piezoelectric element. The oscillators are arranged in a one-dimensional array in an azimuth direction (scanning direction), for example. In the ultrasonic probe 12, the number of oscillators can be appropriately changed in accordance with an object to be diagnosed, and the oscillators may be arranged in a two-dimensional array. A probe of electronic scanning type is used as the ultrasonic probe 12. Any probe such as a convex probe, a linear probe, and a sector probe can be used.

A user uses the above-described ultrasonic probe 12, and brings the transmission/reception surface of the ultrasonic probe 12 for an ultrasonic wave into contact with the surface (skin) of the subject. Although the ultrasonic probe 12 is brought into contact with the surface of the subject here, the ultrasonic probe 12 may be used by being inserting into a body cavity of the subject.

The display 14 displays, for example, an ultrasonic image generated by an image generator 31 (see FIG. 2) of a controller 30. For example, a liquid crystal display, an organic EL display, a CRT display, and a touch panel can be used as the display 14.

The operation input 15 is a user interface for a user to perform an input operation. The operation input 15 converts the input operation performed by the user into operation information, and inputs the operation information to the controller 30. Examples of the operation input 15 include an operation panel including a plurality of input switches, a keyboard, and a mouse. When a touch panel is used as the display 14, the touch panel also functions as a part of the operation input 15.

As illustrated in FIG. 2, the apparatus body 11 includes a transmitter 21, a receiver 22, and a controller 30 in the ultrasonic diagnostic apparatus 10. The controller 30 controls the display 14, the transmitter 21, and the receiver 22. The controller 30 includes the image generator 31, a high-luminance searcher 32, and a determiner 33.

The controller 30 is, for example, an apparatus including a central processing unit (CPU), a read only memory (ROM), and a random access memory (RAM), for example, a computer. Each function of the controller 30 is implemented by the CPU referring to a control program and various pieces of data stored in the ROM or the RAM and executing the control program. Part or all of the functions of the controller 30 may be implemented by a digital signal processor (DSP) or a dedicated hardware circuit.

The transmitter 21 transmits a voltage pulse to the ultrasonic probe 12 in accordance with the controller 30. The transmitter 21 includes, for example, a high frequency pulse oscillator and a pulse adjuster. The high frequency pulse oscillator generates a voltage pulse. The pulse adjuster adjusts the voltage amplitude, pulse width, and transmission timing for the voltage pulse based on a drive signal from the controller 30. The transmitter 21 adjusts the voltage pulse generated by the high frequency pulse oscillator so that the voltage pulse has the voltage amplitude, pulse width, and transmission timing based on the drive signal from the controller 30 in the pulse adjuster, and transmits the voltage pulse to the ultrasonic probe 12. At this time, the transmitter 21 transmits the voltage pulse to an oscillator, which generates an ultrasonic wave, of the ultrasonic probe 12 so that the oscillator is shifted in an azimuth direction (operation direction), thereby performing scanning using an ultrasonic wave.

The receiver 22 performs processing by receiving a reception signal obtained by converting an ultrasonic echo from the ultrasonic probe 12 in accordance with the controller 30. The receiver 22 includes, for example, an amplifier, an A/D conversion circuit, and a phasing addition circuit. The amplifier amplifies the reception signal received from the ultrasonic probe 12 for each oscillator with a preset amplification factor. The A/D conversion circuit converts the amplified reception signal from an analog signal to a digital signal for each oscillator. The phasing addition circuit arranges time phases by giving delay time to the reception signal that has been A/D-converted for each of oscillators, adds (performs phasing addition for) the time phases, and generates sound ray data.

The image generator 31 processes and adjusts the sound ray data transmitted from the receiver 22, and converts the sound ray data into a luminance signal. The luminance signal is in accordance with the signal strength of the reception signal generated at the receiver 22. An ultrasonic image (B mode image) in a brightness (B) mode serving as a tomographic image is generated by performing, for example, coordinate conversion so that the converted luminance signal matches an orthogonal coordinate system. That is, the image generator 31 generates an ultrasonic image of a biotissue inside the subject. The image generator 31 outputs the generated ultrasonic image to the display 14.

With the above-described configuration, the ultrasonic diagnostic apparatus 10 transmits an ultrasonic wave to a subject, receives a reflected wave (ultrasonic echo) of an ultrasonic wave reflected inside the subject, converts the received ultrasonic echo into a reception signal, and generates and displays an ultrasonic image based on the reception signal.

With poor anatomical knowledge for an ultrasonic image, it is unfortunately difficult to visually recognize the expansion of a region into which liquid medicine or saline is injected on an ultrasonic image. Even when the ultrasonic diagnostic apparatuses disclosed in WO 2017/138086 and JP 2006-20800 A are used, it is difficult to determine the region. In the embodiment, the controller 30 thus includes the high-luminance searcher 32 and the determiner 33.

The high-luminance searcher 32 searches for a pixel having a luminance higher than luminance values of pixels presently attracting attention (pixels of attention) in pixels in a predetermined direction of the pixel of attention in relation to pixels constituting an ultrasonic image generated at the image generator 31. If there is a pixel having a luminance higher than a luminance value of the pixel of attention, a high-luminance search result is set to 1, and if not, the high-luminance search result is set to 0. Such a high-luminance search is performed in at least two directions that face each other across the pixel of attention, and is performed for all pixels.

The determiner 33 determines a low-luminance region in an ultrasonic image based on the luminance value of pixels constituting the ultrasonic image. The low-luminance region has a relatively low luminance, and is sandwiched between a plurality of high-luminance regions having relatively high luminance Specifically, if there is a pixel having a luminance higher than a pixel of attention in at least two directions, the pixel of attention is determined as a low-luminance region based on the high-luminance search result in at least the two directions searched at the high-luminance searcher 32. This operation determines a low-luminance region, which is a region of pixels, having a low luminance value, sandwiched between pixels having a high luminance

<Region Display Method and Region Display Program>

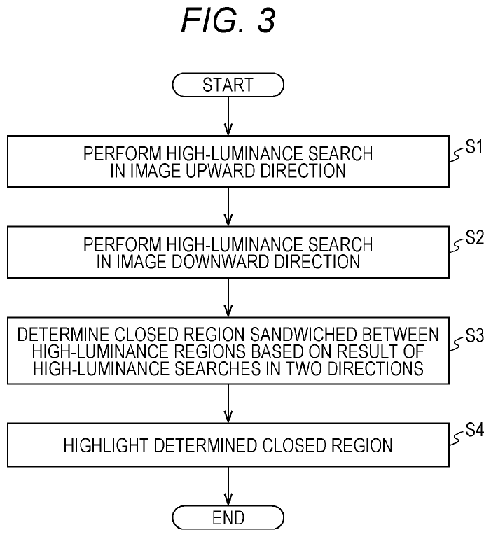

A region display method will now be described with reference to FIGS. 3 to 5D. The region display method is performed by executing a control program (region display program) in the ultrasonic diagnostic apparatus 10 having the above-described configuration.

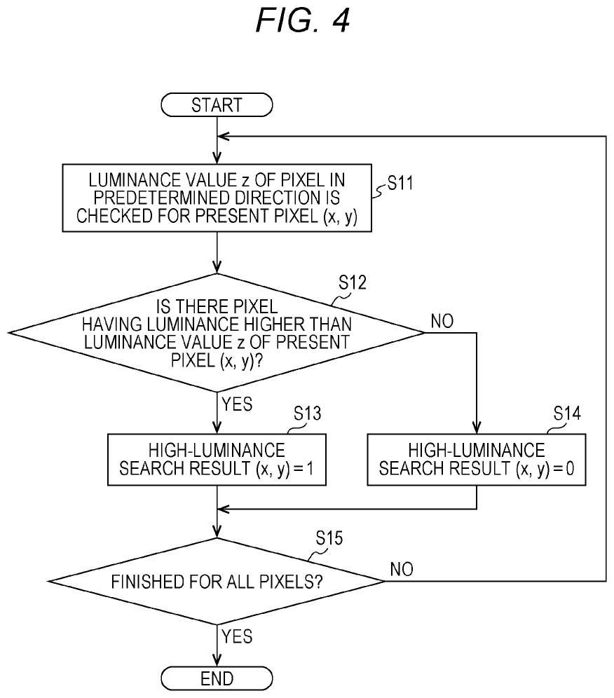

FIG. 3 is a flowchart illustrating a region display method according to the embodiment. FIG. 4 is a flowchart illustrating a high-luminance search in FIG. 3. FIG. 5A schematically illustrates an ultrasonic image before highlighting. FIG. 5B schematically illustrates an ultrasonic image that has been subject to a high-luminance search in an image upward direction. FIG. 5C schematically illustrates an ultrasonic image that has been subject to a high-luminance search in an image downward direction. FIG. 5D schematically illustrates an ultrasonic image in which a low-luminance region determined based on a result of the high-luminance search in two directions is highlighted.

Before a low-luminance region is determined and highlighted as described later, the ultrasonic probe 12 performs measurement, and the image generator 31 generates an ultrasonic image. For example, in one example, as illustrated in FIG. 5A, an ultrasonic image E1 is generated. The ultrasonic image E1 has a high-luminance region H1 on the lower side of the image, a high-luminance region H2 on the upper side of the image, and a low-luminance region L corresponding to a region other than the high-luminance regions H1 and H2. In the ultrasonic image E1 and later-described ultrasonic images E2 to E4 (see FIGS. 5B to 5D), the lower side of the image corresponds to a position deep from the surface of a subject, and the upper side of the image corresponds to a position shallow from the surface of the subject.

(Step S1)

The high-luminance searcher 32 performs a high-luminance search in an image upward direction on the ultrasonic image E1 generated at the image generator 31 for pixels constituting the ultrasonic image E1. The high-luminance search is performed in the procedure in FIG. 4. The high-luminance search in the image upward direction will be described with reference to FIGS. 4 and 5B.

(Step S11)

A luminance value z of a pixel on a line in the image upward direction, which is a predetermined direction, is checked for a present pixel (x, y) presently attracting attention. Here, (x, y) are coordinates on the ultrasonic image, and the upper left of the image is set as the origin (0, 0). The image upward direction is a direction on the upward side of the image from the present pixel (x, y), that is, a direction from a deep position to a shallow position of the surface of the subject. For example, assuming that the number of pixels of the ultrasonic image E1 is m×n, first, the luminance value z from a pixel (0, n−2) to the pixel (0, 0) is checked for the present pixel (0, n−1).

(Step S12)

It is checked whether or not there is a pixel having a luminance higher than the luminance value z of the present pixel (x, y) in pixels in the image upward direction. If there is such a pixel (YES), the processing proceeds to Step S13. If there is not such a pixel (NO), the processing proceeds to Step S14.

(Step S13)

If there is a pixel having a luminance higher than the luminance value z of the present pixel (x, y) in the pixels in the image upward direction, a high-luminance search result (x, y) for the present pixel (x, y) is set to 1. For example, if the pixel (i, j1) in FIG. 5B is the present pixel presently attracting attention, a high-luminance search result (i, j1)=1 holds since there are pixels constituting the high-luminance region H2 on the upper side of the image than the present pixel (i, j1).

(Step S14)

If there is not a pixel having a luminance higher than the luminance value z of the present pixel (x, y) in the pixels in the image upward direction, the high-luminance search result (x, y) for the present pixel (x, y) is set to 0. For example, if the pixel (i, j2) in FIG. 5B is the present pixel presently attracting attention, a high-luminance search result (i, j2)=0 holds since there are not pixels having high luminance on the upper side of the image than the present pixel (i, j2).

(Step S15)

It is checked whether or not the procedure illustrated above in Steps S11 to S14 has been finished for all pixels. If the procedure has not been finished for all the pixels (NO), the processing returns to Step S11. If the procedure has been finished (YES), the procedure in Steps S11 to S14 is finished, and the processing proceeds to Step S2.

In Step S15, the high-luminance search result in the image upward direction is acquired for all the pixels. Assuming that the number of pixels of the ultrasonic image E1 is m×n, first, the luminance value z from the pixel (0, n−2) to the pixel (0, 0) is checked for the pixel (0, n−1), and a high-luminance search result (0, n−1) is acquired. The luminance value z from a pixel (0, n−3) to the pixel (0, 0) is then checked for the pixel (0, n−2), and a high-luminance search result (0, n−2) is acquired. After that, the procedure in Steps S11 to S14 is repeated until x=m−1 and y=0 are established, whereby the high-luminance search results in the image upward direction are acquired for all the pixels.

The ultrasonic image E2 in FIG. 5B can be acquired by the procedure in Steps S11 to S15 of Step S1. In the ultrasonic image E2, a low-luminance region LU1 (see lower right shaded part in FIG. 5B) is determined from the low-luminance region L of the ultrasonic image E1. There are pixels having a high luminance in the upper part of the low-luminance region LU1. The low-luminance region LU1 can be distinguished from a low-luminance region LU2. There is not a pixel having a high luminance in the upper part of the low-luminance region LU2.

(Step S2) The high-luminance searcher 32 performs a high-luminance search in an image downward direction on the ultrasonic image E1 generated at the image generator 31 for pixels constituting the ultrasonic image E1. The high-luminance search is performed in the procedure in FIG. 4 similarly to Step S1. The high-luminance search in the image downward direction will be described with reference to FIGS. 4 and 5C.

(Step S11) For a present pixel (x, y) presently attracting attention, a luminance value z of a pixel on a line in the image downward direction, which is a predetermined direction, is checked. The image downward direction is a direction from the present pixel (x, y) to the lower side of the image, that is, a direction from a shallow position to a deep position of the surface of the subject. Assuming that the number of pixels of the ultrasonic image E1 is m×n, first, the luminance value z from a pixel (0, 1) to the pixel (0, n−1) is checked for the present pixel (0, 0).

(Step S12)

It is checked whether or not there is a pixel having a luminance higher than the luminance value z of the present pixel (x, y) in pixels in the image downward direction. If there is such a pixel (YES), the processing proceeds to Step S13. If there is not such a pixel (NO), the processing proceeds to Step S14.

(Step S13) If there is a pixel having a luminance higher than the luminance value z of the present pixel (x, y) in the pixels in the image downward direction, a high-luminance search result (x, y) for the present pixel (x, y) is set to 1. For example, if the pixel (i, j3) in FIG. 5C is defined as the present pixel presently attracting attention, a high-luminance search result (i, j3)=1 holds since there are pixels constituting the high-luminance region H2 on the lower side of the image than the present pixel (i, j3).

(Step S14)

If there is not a pixel having a luminance higher than the luminance value z of the present pixel (x, y) in the pixels in the image downward direction, the high-luminance search result (x, y) for the present pixel (x, y) is set to 0. For example, if a pixel (i, j4) in FIG. 5C is defined as the present pixel presently attracting attention, a high-luminance search result (i, j4)=0 holds since there are not pixels having a high luminance on the lower side of the image than the present pixel (i, j4).

(Step S15)

It is checked whether or not the procedure illustrated above in Steps S11 to S14 has been finished for all pixels. If the procedure has not been finished for all the pixels (NO), the processing returns to Step S11. If the procedure has been finished (YES), the procedure in Steps S11 to S14 is finished, and the processing proceeds to Step S3.

In Step S15, the high-luminance search result in the image downward direction is acquired for all the pixels. Assuming that the number of pixels of the ultrasonic image E1 is m×n, first, the luminance value z from the pixel (0, 1) to the pixel (0, n−1) is checked for the pixel (0, 0), and a high-luminance search result (0, 0) is acquired. The luminance value z from a pixel (0, 2) to the pixel (0, n−1) is then checked for the pixel (0, 1), and a high-luminance search result (0, 1) is acquired. After that, the procedure in Steps S11 to S14 is repeated until x=m−1 and y=n−1 are established, whereby the high-luminance search results are acquired for all the pixels.

An ultrasonic image E3 in FIG. 5C can be acquired by the procedure in Steps S11 to S15 of Step S2. In the ultrasonic image E3, a low-luminance region LD1 (see lower left shaded part in FIG. 5C) is determined from the low-luminance region L of the ultrasonic image E1. There are pixels having a high luminance in the lower part of the low-luminance region LD1. The low-luminance region LD1 can be distinguished from a low-luminance region LD2. There is not a pixel having a high luminance in the lower part of the low-luminance region LD2.

(Step S3)

The determiner 33 determines a closed region based on the high-luminance search results in two directions. The closed region is a low-luminance region sandwiched between high-luminance regions. Specifically, the closed region is determined by determining the logical product (AND) of the high-luminance search result in the image upward direction and the high-luminance search result in the image downward direction. The former high-luminance search result is acquired in the above-described Step S2. The latter high-luminance search result is acquired in the above-described Step S3.

More specifically, the product of the high-luminance search result (0 or 1) in the image upward direction and the high-luminance search result (0 or 1) in the image downward direction is determined for each pixel. At this time, a pixel, for which high-luminance search results both in the image upward direction and in the image downward direction is 1, has a product of 1×1=1. A pixel, for which a high-luminance search result in one or both of the image upward direction and the image downward direction is 0, has a product of 1×0=0, 0×1=0, or 0×0=0. As a result, a pixel having a product of 1 is determined as the closed region.

An ultrasonic image E4 in FIG. 5D can be acquired by the procedure in Step S3. In the ultrasonic image E4, a closed region C can be determined from the low-luminance region L of the ultrasonic image E1. The closed region C is a low-luminance region sandwiched between the high-luminance region H1 and the high-luminance region H2 in the vertical direction of the image.

(Step S4)

The display 14 displays the ultrasonic image E4 such that the determined closed region C is highlighted in a display mode different from that for other regions (high-luminance regions H1 and H2 and low-luminance region LE1). The low-luminance region LE1 is a low-luminance region other than the closed region C. The display 14 highlights the closed region C on the ultrasonic image E4 such that the regions other than the closed region C (high-luminance regions H1 and H2 and low-luminance region LE1) are painted a display color (display mode) different from that of the closed region C, for example. With the above, a series of procedures is finished.

In the embodiment, the above-described procedures determine the closed region C, which interests a user, in low-luminance regions, thereby improving the determination accuracy. The determined closed region C is highlighted so that the visibility of the closed region C can be improved.

For example, liquid medicine and saline are injected into a biotissue between a plurality of muscle tissues in HydroRelease. With poor anatomical knowledge for an ultrasonic image, the expansion of an injection region sometimes cannot be visually recognized in the ultrasonic image. In contrast, in the embodiment, the above-described procedures determine the biotissue between muscle tissues as the closed region C, and highlights the closed region C, thereby improving the visibility of the closed region C. The embodiment can be applied not only to a biotissue between a plurality of muscle tissues, but to a biotissue in a bladder and a biotissue in a tumor.

Although, in the above-described Steps S1 and S2, the high-luminance searcher 32 determines the closed region C, which is a low-luminance region sandwiched between the plurality of high-luminance regions H1 and H2, in a subject depth direction, which corresponds to the vertical direction of the ultrasonic image E4, the high-luminance searcher 32 may determine the closed region C not only in the vertical direction but in another direction. For example, a closed region, which is a low-luminance region sandwiched between a plurality of high-luminance regions, may be determined in a direction along the surface of the subject, which is the horizontal direction of the ultrasonic image E4. The closed region, which is a low-luminance region surrounded by a plurality of high-luminance regions, may be determined in the vertical direction and the horizontal direction of the ultrasonic image E4. The closed region may be determined not only in the vertical and horizontal directions but in any direction.

Although the high-luminance searcher 32 performs high-luminance search for the ultrasonic image E1 generated by the image generator 31 in the above-described Steps S1 and S2, image processing such as noise removing processing for the ultrasonic image E1 may be performed before the processing. For example, noise removing processing such as smoothing processing and morphology processing is performed.

Although the high-luminance searcher 32 uses two values of 1 or 0 as a high-luminance search result in the above-described Steps S1 and S2, the two values are not limitative. Multiple values such as the difference between the luminance value of a present pixel and the luminance value of a pixel having a high luminance may be used.

Although the determiner 33 determines a logical product of high-luminance search results in two directions in the above-described Step S3, the determiner 33 may determine the closed region C not by determining the logical product but by performing weighting in each direction and adding a result after the weighting.

For example, a weight coefficient in an upper direction is set to 1.5, and a weight coefficient in a lower direction is set to 1.0 in the vertical direction. Weighting is performed more heavily in the upper direction than in the lower direction. A result after the weighting is added. The closed region C is thereby determined. The weight coefficient in the upper direction is set to 1.5, the weight coefficient in the lower direction is set to 1.0, and a weight coefficient in a horizontal direction is set to 0.5 in the vertical and horizontal directions. Weighting is performed more heavily in the lower direction than in the horizontal direction and more heavily in the upper direction than in the lower direction. A result after the weighting is added. The closed region C is thereby determined.

Usually, an ultrasonic diagnostic apparatus has higher detection accuracy for a biotissue in the vertical direction than in the horizontal direction, and higher detection accuracy for a biotissue in the upper direction than in the lower direction. Thus, the determination accuracy for the closed region C can be improved by increasing a weight coefficient in the vertical direction or the upper direction.

If a plurality of closed regions C is determined, a closed region C determined near the center of the ultrasonic image E4 may be preferentially determined as a closed region C that interests a user. This is because the ultrasonic probe 12 is brought into contact with the surface of the subject such that a region that interests the user comes close to the center of the ultrasonic image at the time of ultrasonic diagnosis. This can improve the determination accuracy for the closed region C that interests the user.

[Variation 1]

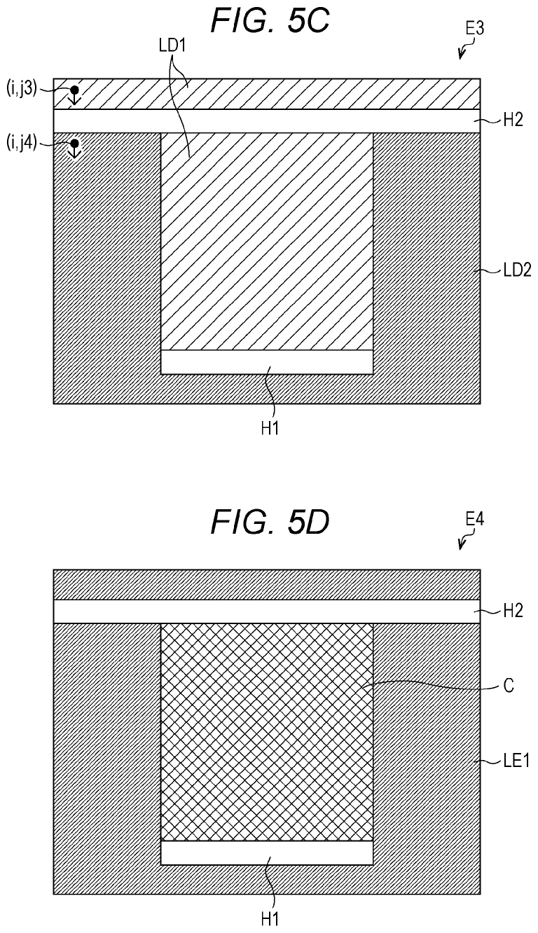

Variations of the embodiment will be described with reference to FIGS. 6 to 7C.

FIG. 6 is a flowchart illustrating a variation of a region display method according to the embodiment. FIG. 7A schematically illustrates an ultrasonic image in which a low-luminance region is highlighted at a time t1 immediately before a time t2. FIG. 7B schematically illustrates an ultrasonic image in which a low-luminance region is highlighted at the time t2. FIG. 7C schematically illustrates an ultrasonic image in which a changed region is highlighted, the changed region being a region, in which the luminance value has been changed between the time t1 and the time t2, in the low-luminance region.

In the variation, an ultrasonic diagnostic apparatus is required to have configuration similar to that of the ultrasonic diagnostic apparatus 10 in FIGS. 1 and 2. A region display method also has the procedure described in FIGS. 3 and 4 in the first half part. Consequently, here, the description overlapping with the above description will be omitted, and a region display method in the variation will be described.

(Steps S1 to S3)

Steps S1 to S3 are as described above. As illustrated in FIG. 7A, an ultrasonic image E4(t1) at the time t1 immediately before the present time t2 is acquired. As illustrated in FIG. 7B, an ultrasonic image E4(t2) at the present time t2 is acquired.

In the ultrasonic image E4(t1), as illustrated in FIG. 7A, a closed region C(t1) is determined. The closed region C(t1) is a low-luminance region sandwiched between a high-luminance region H1(t1) and a high-luminance region H2(t1) in the vertical direction of the image. A low-luminance region LE1(t1) is a low-luminance region other than the closed region C(t1). In the ultrasonic image E4(t2), as illustrated in FIG. 7B, a closed region C(t2) is determined. The closed region C(t2) is a low-luminance region sandwiched between a high-luminance region H1(t2) and a high-luminance region H2(t2) in the vertical direction of the image. A low-luminance region LE1(t2) is a low-luminance region other than the closed region C(t2).

(Step S5)

The determiner 33 sets the ultrasonic image E4(t1) at the time t1 as a previous frame, and sets the ultrasonic image E4(t2) at the present time t2 as a present frame. The determiner 33 determines a difference region D in which temporal change of a luminance value occurs between the closed region C(t1) of the previous frame and the closed region C(t2) of the present frame. Specifically, the difference region D in FIG. 7C is determined by determining the difference in luminance values between the closed region C(t1) in FIG. 7A and the closed region C(t2) in FIG. 7B.

(Step S6)

The display 14 displays an ultrasonic image E5 such that the determined difference region D is highlighted in a display mode different from that for other regions (high-luminance regions H1 and H2 and low-luminance region LE2). The low-luminance region LE2 is a low-luminance region other than the difference region D. The display 14 highlights the difference region D on the ultrasonic image E5 such that the regions other than the difference region D (high-luminance regions H1 and H2 and low-luminance region LE2) are painted a display color (display mode) different from that of the difference region D, for example. With the above, a series of procedures is finished.

When the difference region D is highlighted, the degree at which the display color of the difference region D is varied (degree of change), that is, the degree of highlight of the display color may be changed in accordance with the temporal change of the luminance value in the difference region D. For example, when the temporal change of the luminance value is large, the degree of highlight of the display color is increased. When the temporal change of the luminance value is small, the degree of highlight of the display color is decreased.

When the temporal change of the luminance value is stopped, the degree of highlight of the display color may be reduced over time. An afterimage is thereby displayed for the highlighted difference region D. Although the continuous same highlights may decrease the visibility of a user, such afterimage display can improve the visibility.

In the variation, the difference region D, in which the luminance value is changed in a low-luminance region that interests a user, is determined and highlighted by the above-described procedure, so that the visibility of the difference region D can be improved. The high-luminance searcher 32 and the determiner 33 determine the closed region C and the difference region D by simple arithmetic processing. Consequently, processing can be performed without lowering a frame rate, and the difference region D can be displayed at a high frame rate.

Although the determiner 33 determines the difference region D, in which the temporal change of the luminance value occurs, from the difference between the present frame at the present time t2 and the previous frame at the time t1 immediately before the present time t2 in the above-described Step S5, the previous frame is not required to be the previous frame at the time t1 immediately before the present time t2. For example, a frame at a time before the time t1 immediately before the present time t2, that is, a frame several frames before may be set as the previous frame. A frame of the ultrasonic image estimated from the reference or standard frame may be set as the previous frame.

[Variation 2]

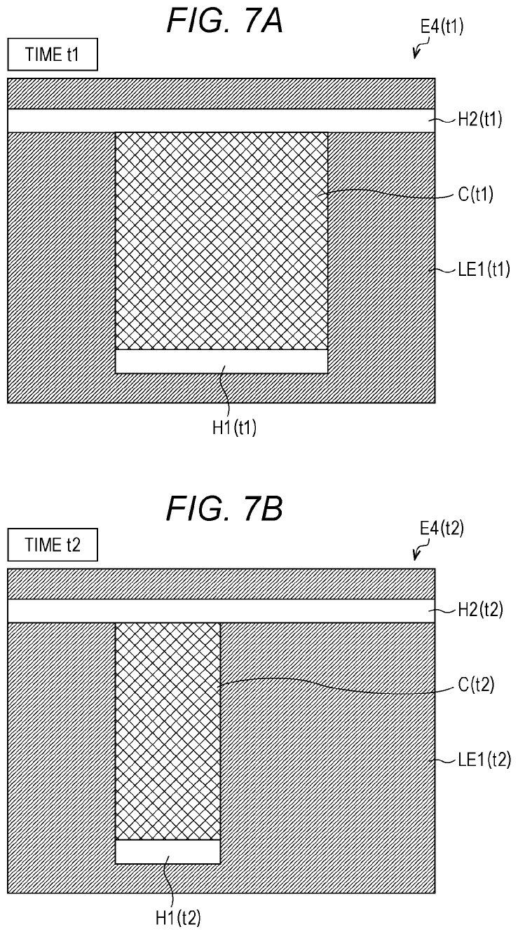

Another variation of the embodiment will be described with reference to FIGS. 2 and 8. FIG. 8 schematically illustrates a screen of the display in which an ultrasonic image and an operation region are displayed, a low-luminance region being highlighted in the ultrasonic image, the operation region being used for changing highlight gain and color.

In the variation, the controller 30 further includes a display mode changer 34 that changes the display mode of the closed region C based on designation from a user (see FIG. 2).

The display mode changer 34 gives an instruction to change the display mode of the closed region C determined by the determiner 33. Instruction information on the display mode of the closed region C, for example, a display color or the degree of highlight of the display color is given to the display 14 based on the operation information input by the user to the operation input 15. In this case, as illustrated in FIG. 8, the display 14 displays a gain operation region 141 and a color operation region 142 on a screen 140 together with the ultrasonic image E4.

The gain operation region 141 (second display mode changer) is used at the time when gain (sensitivity) is set. The gain indicates the degree of highlight with respect to the luminance value of each pixel in the closed region C. For example, when the gain operation region 141 is clicked with a mouse, a gain input region is displayed, and the degree of highlight is changed based on the input gain. The gain may be a numerical value such as a threshold value, or may be selected from a plurality of levels such as weak, standard, and strong.

The color operation region 142 (first display mode changer) is used at the time when a display color for highlighting the closed region C is set. For example, when the color operation region 142 is clicked with a mouse, a display color input region is displayed, and the highlighting display color is changed based on the input display color. For example, color other than that used in the ultrasonic image E1 is required to be selectable as the highlighting display color. For example, when black and white are used in the ultrasonic image E1, color other than black and white is required to be selectable. At this time, not only the hue of display color but saturation and lightness may be selectable.

The display 14 highlights the determined closed region C based on the instruction information given from the display mode changer 34 such that, for example, the display color of the closed region C determined at the determiner 33 is different from that of the region other than the closed region C (high-luminance regions H1 and H2 and low-luminance region LE1).

The display mode changer 34 allows the user to select highlight sensitivity and effect color.

[Variation 3]

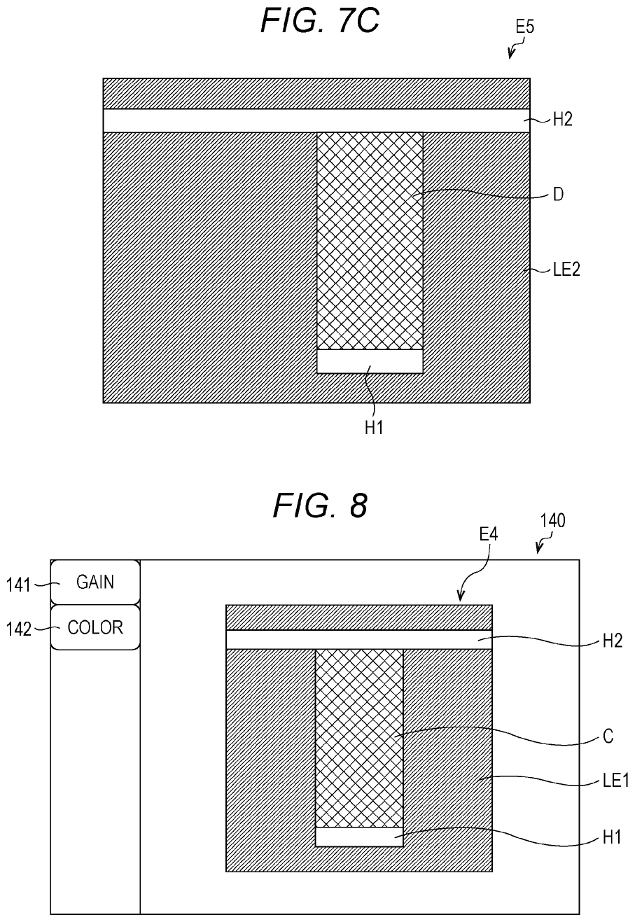

Another variation of the embodiment will be described with reference to FIG. 9. FIG. 9 schematically illustrates a screen of the display in which an ultrasonic image before highlighting and an ultrasonic image after a low-luminance region is highlighted are displayed side by side.

In the variation, the display 14 displays the ultrasonic image E1 on the screen 140 while displaying the ultrasonic image E4 on the screen 140. For example, as illustrated in FIG. 9, two of the ultrasonic images E1 and E4 are displayed side by side. That is, the ultrasonic image E1 before determining the closed region C and the ultrasonic image E4 after determining the closed region C are displayed side by side.

In this way, the ultrasonic image E1 and the ultrasonic image E4 are simultaneously displayed. The ultrasonic image E1 is an image before the closed region C is determined. In the ultrasonic image E4, the closed region C after the closed region C is determined has a display mode different from that of a region other than the closed region C. Consequently, even if the closed region C is erroneously determined, the user can check the ultrasonic image E1 before the closed region C is determined, so that the risk related to visual recognition can be reduced.

[Variation 4]

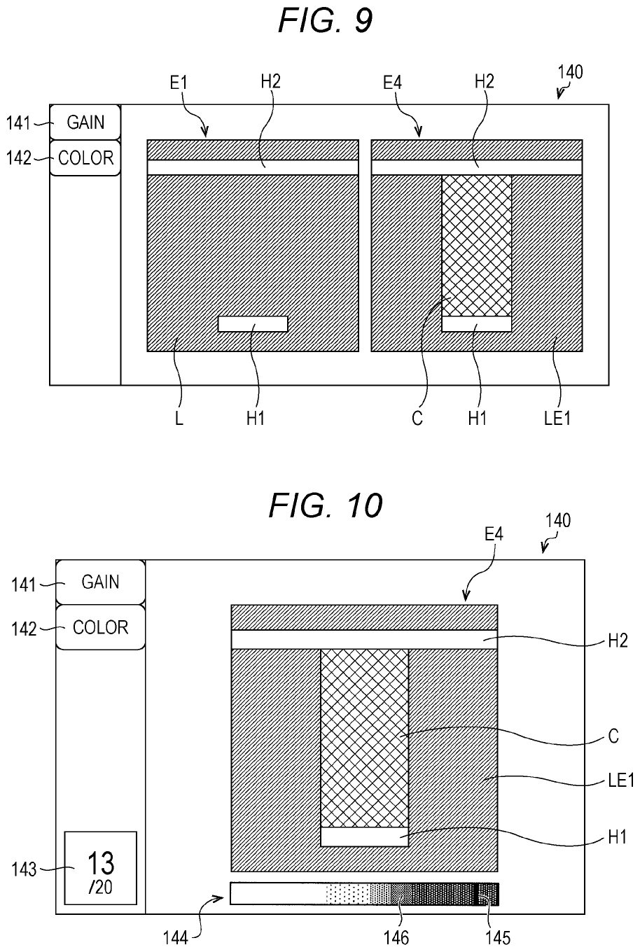

Another variation of the embodiment will be described with reference to FIG. 10. FIG. 10 schematically illustrates a screen of the display in which an area information display region and a seek bar are displayed, the area information display region indicating an area value of a highlighted low-luminance region, the seek bar relating to the presently displayed ultrasonic image.

In the variation, the display 14 displays the ultrasonic image E4 on the screen 140 while displaying an area information display region 143 on the screen 140. The area information display region 143 has area information indicating the area value of the determined closed region C. For example, as illustrated in FIG. 10, the area information display region 143 displays the area value of the closed region C with respect to the total area value of the ultrasonic image E4 in a format of numerical value. Instead of the format of numerical value, the area information may be displayed in a format of graph such as a bar graph and a pie chart.

The user can check the state of the closed region C semi-quantitatively (as a numerical value that correlates with a quantitative numerical value) based on a numerical value and a graph of the area value of the closed region C displayed on the area information display region 143. For example, when the closed region C is a biotissue between a plurality of muscle tissues, and liquid medicine is injected into the biotissue, an injection amount of the liquid medicine can be semi-quantitatively checked. When the closed region C is a biotissue in a bladder, and urine is discharged from the bladder, the injection amount of the urine can be semi-quantitatively checked. When the closed region C is a biotissue in a tumor, and the therapeutic effect on the tumor is checked, the area occupied by the tumor can be semi-quantitatively checked.

In the variation, the display 14 displays a seek bar 144 (time bar) on the screen 140. The seek bar 144 indicates the time position of the presently displayed ultrasonic image E4 with a slider 145 on a time axis. The display 14 also displays a gradation region 146 on the seek bar 144. The gradation region 146 indicates gradation correlated with the magnitude of the area value of the closed region C in the ultrasonic image E4 at each time position. In the gradation region 146, concentration display is changed from a low concentration to a high concentration in proportion to the size of the area value such that the closed region C with a small area value has a low concentration and the closed region C with a large area value has a high concentration.

The user moves the slider 145 along the time axis with reference to the gradation of the gradation region 146 displayed on the seek bar 144, and displays the ultrasonic image E4 including the closed region C with a desired area on the screen 140. The closed region C can thereby be checked.

The above-described variations 2 to 4 may be applied not only to the closed region C but to the difference region D in the variation 1.

Although embodiments of the present invention have been described and illustrated in detail, the disclosed embodiments are made for purposes of illustration and example only and not limitation. The scope of the present invention should be interpreted by terms of the appended claims That is, the present invention can be carried out in various forms without departing from the gist or the main features of the invention.

What is claimed is:

1. A medical image display apparatus comprising:
a hardware processor that determines a low-luminance region on a medical image related to a biotissue inside a subject based on a luminance value of a pixel constituting the medical image, the low-luminance region having a relatively low luminance and being sandwiched between a plurality of high-luminance regions having a relatively high luminance; and
a display that displays the determined low-luminance region on the medical image in a display mode different from that of a region other than the low-luminance region.

2. The medical image display apparatus according to claim 1,
wherein, when there is a pixel having a higher luminance than a pixel in at least two directions facing each other across the pixel, the hardware processor determines the latter pixel as the low-luminance region.

3. The medical image display apparatus according to claim 1,
wherein the low-luminance region is sandwiched between the plurality of high-luminance regions in a depth direction of the subject on the medical image.

4. The medical image display apparatus according to claim 1,
wherein the low-luminance region is sandwiched between the plurality of high-luminance regions in a direction along a surface of the subject on the medical image.

5. The medical image display apparatus according to claim 1,
wherein the display mode means display color.

6. The medical image display apparatus according to claim 1,
wherein the hardware processor further determines a changed region, in which temporal change of a luminance value has occurred, in the low-luminance region, and
the display displays the determined changed region in a display mode different from that of the low-luminance region other than the changed region.

7. The medical image display apparatus according to claim 6,
wherein the display displays the determined changed region in display color different from that of the low-luminance region other than the changed region.

8. The medical image display apparatus according to claim 7,
wherein the display changes a degree at which the display color of the changed region is varied in accordance with temporal change of the luminance value in the changed region.

9. The medical image display apparatus according to claim 8,
wherein, when temporal change of the luminance value in the changed region is stopped, the display reduces, over time, a degree at which the display color of the changed region is varied.

10. The medical image display apparatus according to claim 1, wherein the hardware processor changes the display mode based on designation from a user.

11. The medical image display apparatus according to claim 1,
wherein the display mode means display color, and
the hardware processor changes sensitivity of a degree at which the display color is varied with respect to a luminance value of a pixel in the determined low-luminance region based on designation from a user.

12. The medical image display apparatus according to claim 1,
wherein the hardware processor determines the low-luminance region near a center of the medical image.

13. The medical image display apparatus according to claim 1,
wherein the display simultaneously displays the medical image before the low-luminance region is determined and the medical image in which the low-luminance region after the low-luminance region is determined is displayed in a display mode different from that of a region other than the low-luminance region.

14. The medical image display apparatus according to claim 1,
wherein the display displays area information indicating an area value of the low-luminance region.

15. The medical image display apparatus according to claim 14,
wherein the area information indicates an area value of the low-luminance region in a numerical value format or a graph format.

16. The medical image display apparatus according to claim 14,
wherein the display displays a seek bar indicating a time position of the presently displayed medical image on a time axis while displaying gradation correlated with magnitude of the area value on the seek bar.

17. A region display method comprising:
determining a low-luminance region on a medical image related to a biotissue inside a subject based on a luminance value of a pixel constituting the medical image, the low-luminance region having a relatively low luminance and being sandwiched between a plurality of high-luminance regions having a relatively high luminance; and
displaying the determined low-luminance region on the medical image in a display mode different from that of a region other than the low-luminance region.

18. A non-transitory recording medium storing a computer readable region display program causing a computer to execute:
determining a low-luminance region on a medical image related to a biotissue inside a subject based on a luminance value of a pixel constituting the medical image, the low-luminance region having a relatively low luminance and being sandwiched between a plurality of high-luminance regions having a relatively high luminance; and
displaying the determined low-luminance region on the medical image in a display mode different from that of a region other than the low-luminance region.
